(19) United States
(12) Patent Application Publication
(10) Pub. No.: US 2010/0260903 A1
(43) Pub. Date: Oct. 14, 2010

(54) DEVICE FOR THE QUALIFICATION OF COOKING OILS, AND METHODS

(76) Inventors: Ai-Ping . Wei, Woodbury, MN (US); Raj Rajagopal, Woodbury, MN (US); Catherine Bineau, Asnieres sur Seine (FR)

Correspondence Address: 3M INNOVATIVE PROPERTIES COMPANY, PO BOX 33427, ST. PAUL, MN, 55133-3427, US

(21) Appl. No.: 12/521,803

(22) PCT Filed: Jan. 4, 2008
(86) PCT No.: PCT/US08/50172

§ 371 Date: Jun. 10, 2010

Related U.S. Application Data
(60) Provisional application No. 60/883,868, filed on Jan. 8, 2007.

(51) Int. Cl. G01N 33/03; G01N 33/26; G01N 21/64; G01J 1/42

(52) U.S. Cl. 426/231; 250/459.1; 250/458.1

(57) ABSTRACT

A method of determining the quality of oil by irradiating the oil at a first wavelength, e.g., of about 470 nm, measuring a level of fluorescence of the oil at a second wavelength, e.g., of about 520 nm, comparing the measured fluorescence level to a predetermined threshold level to determine whether or not the oil quality is acceptable. The oil is preferably discarded if the measured fluorescence level exceeds the predetermined threshold level, which can be generally dependent on the oil composition. The fluorescence is correlated to the level of polar components in the oil. Also described are devices for determining the quality of oil via fluorescence.

### TECHNICAL FIELD

This disclosure relates to methods of determining the quality of cooking oils, and devices for those methods.

### BACKGROUND

Fast food restaurants and other kitchens use both vegetable shortenings and animal fats for cooking, usually for frying. Since this operation is carried out at high temperatures, usually about 180° C., often in the presence of water, oxygen and starch, several chemical changes take place in the oil, degrading the oil quality. The quality of frying oil is of increasing concern because prolonged exposure to high temperatures could create a variety of new substances or compounds such as acrylamides, polymers, radicals, free fatty acids, and polar compounds. Some of these compounds are suspect in some health conditions, such as hypertension heart attacks, and diabetes.

In some restaurants or kitchens, the decision to change or not to change the oil is based on a visual inspection of the color of the oil or the level of particulates present in the oil. Known methods to more accurately monitor the quality of the oil can be expensive, time consuming, and can also depend strongly on, for example, the temperature of the oil during measurement. The test can then lead to erroneous results, resulting in either discarding good oil, or retaining degraded oil. Prior to this disclosure, there has been no systematic and accurate way to monitor oil quality quickly and easily, as the oil is repeatedly used in frying.

Improvements in measuring oil quality are needed.

### SUMMARY OF THE DISCLOSURE

The present disclosure is directed to methods for measuring the quality of cooking oil and device that use those methods. Generally, the methods include irradiating the oil at a first wavelength, measuring a level of fluorescence of the oil at a second wavelength different than the first wavelength, determining whether or not the oil is acceptable. The level of fluorescence can be a correlation to the level of polar components present in the oil.

In one particular aspect, this disclosure is directed to a method of determining the quality of oil, the method comprising irradiating the oil at a first wavelength, e.g., of about 470 nm, measuring a level of fluorescence of the oil at a second wavelength, e.g., of about 520 nm, comparing the measured fluorescence level to a predetermined threshold level to determine whether or not the oil quality is acceptable. The oil is preferably discarded if the measured fluorescence level exceeds the predetermined threshold level, which can be generally dependent on the oil composition.

The method may be done without contacting the oil, by removing a sample from a larger batch and contacting the sample, or by contacting the larger batch.

A fluorescent marker may be added the oil, which is generally done after removing a sample from the larger batch.

In another particular aspect, this disclosure is directed to a portable (handheld or countertop) device for real-time measurement of the quality of oil. The device includes a means for irradiating the oil at a first wavelength, e.g., 470 nm or a blue light, a means for measuring a level of fluorescence of the oil at a second wavelength, e.g., 520 nm or a green light, and a display.

The means for measuring the level of fluorescence might be an optical sensor or a physical (contact) sensor, such as a swab or a probe.

The device may be configured with a data communication connection to be connected to a data network for storing, retrieving and updating data corresponding to the quality of the oil. Additionally or alternately, the device may be connected to a printer.

These and various other features which characterize the packages of this disclosure are pointed out with particularity in the attached claims. For a better understanding of the packages of the disclosure, their advantages, their use and objectives obtained by their use, reference should be made to the drawings and to the accompanying description, in which there is illustrated and described preferred embodiments of the invention of this disclosure.

### BRIEF DESCRIPTION OF THE DRAWINGS

FIG. 1 is a schematic perspective view of a counter-top device of the present invention for testing the quality of oil;

FIG. 2 is a schematic perspective view of a hand-held device of the present invention for testing the quality of oil;

FIG. 3 is a graphical representation of the fluorescence spectra of Examples 1 and 2;

FIG. 4 is a graphical representation of the fluorescence spectra of Examples 3, 4 and 5; and

FIG. 5 is a graphical representation of the fluorescence spectra of Examples 14, 15 and 16.

### DETAILED DESCRIPTION

The present disclosure is directed to methods of determining the quality of cooking oils, which can be based on the level of polarity or polar compounds present in the oil, and devices for determining the quality of the oil. Examples of common cooking oils include vegetable oils such as corn oil, soybean oil, canola oil, safflower oil, olive oil, palm oil, rapeseed oil, sunflower seed oil, and cottonseed oil.

The methods of this disclosure correlate the level of fluorescence of the oil with the quality of, and continued ability to use, the oil. In some embodiments, the level of fluorescence of the oil correlates to the polar content of the cooking oil, which increases as the quality decreases. Measuring the fluorescence level can thus provide a qualitative, and quantitative, level of cooking oil quality, either based on the polar content or the autofluorescence of the oil.

As cooking oil is repeatedly used, its quality decreases. Decreased quality can lead to decreased taste, odor, and nutrition of the cooked food item. Polar compounds are degradation products formed during cooking in fats and oils, and are proportional to the deterioration of those fats and oils. A common standard method for the determination of the content of polar compounds in animal and vegetable fats and oils is with ISO 8420 “Animal and vegetable fats and oils—Determination of content of polar compounds.” There are various devices available for testing the quality of cooking oil.

Traditional methods for evaluating degraded oil quality use, e.g., dielectric constant measurements, visible and infrared spectroscopies, Fourier transform infrared (FTIR), column chromatography, and ultrasonic techniques. Absorbtive membranes and surface acoustic waves (SAW) have also been used to measure oil quality. These methods, however, are tedious, time consuming, and are not amenable to on-line testing or assessment. For some of these methods, samples of the oil are sent to a remote lab for testing. The devices of the present disclosure provide real-time, on-line testing in a convenient form.

The devices of the present disclosure are readily portable, hand-held devices or countertop devices. In most embodiments, the devices are less than 5 pounds in weight (about 2.2 kg), often less than 3 pounds (about 1.4 kg). Hand-held devices are usually no larger than about 12 inches (about 30 cm) in their largest dimension, often no more than about 8 inches (20 cm). Counter-top devices can be larger than hand-held devices.

The testing devices of the present disclosure are configured to determine the quality of cooking oil (e.g., frying oil) in an easy and real-time manner. The devices measure the fluorescence of the cooking oil, which correlates to the level of polar compounds in the oil, and compare the fluorescence to a predetermined curve or threshold.

In some embodiments, the device is brought into operational contact with the oil to be tested, and the oil is excited or irradiated by radiation. In most embodiments, this radiation is visible light. Visible light having a wavelength of 470 nm is a preferred wavelength for irradiating the oil to be tested, particularly if no fluorescent markers are used. The device then measures the fluorescence level, at a wavelength different than the irradiating wavelength. If wavelengths of 470 nm are used for the irradiating, a preferred measuring wavelength is 520 nm. Different radiation is desired, to eliminate the opportunity for back scatter and background noise.

The device of the present disclosure, for testing the quality of cooking oil via fluorescence, generally includes an informational display, to advise the user of the quality of the tested oil. For example, the device may include a series of LEDS. Separate LEDs may light as the quality of the oil increases.

As another example, the display may include a green light to indicate the oil sample is still acceptable and a red light to indicate the oil should no longer be used. Yellow and/or orange lights may be present between the green light and red light to indicate a progression. Alternately, simple symbols, such as a smiling face and a frowning face, and increments therebetween, could be used. The display may be a quantitative display, providing a specific number of, e.g., polar constituents, in the oil, or estimated percentage of oil left remaining.

The device of the present disclosure may be configured for connection to a data network for storing, retrieving and updating data corresponding to the quality of the oil. Additionally or alternately, the device may be configured for connection to a printer or other output device.

After the testing described in the foregoing has indicated that the oil should no longer be used, the oil can either be discarded, or treated for reuse by one of many techniques known in the art. Physical, chemical and mechanical methods can be used to rejuvenate the oil. Examples of such methods include filtration (e.g. FMC Food Tech, Chicago, Ill.), ionic rejuvenation (Rejuvenoil, Hoei America, Inc., Buffalo Grove, Ill.) and chemical treatment (e.g. U.S. Pat. Nos. 5,391,385 and 6,187,355).

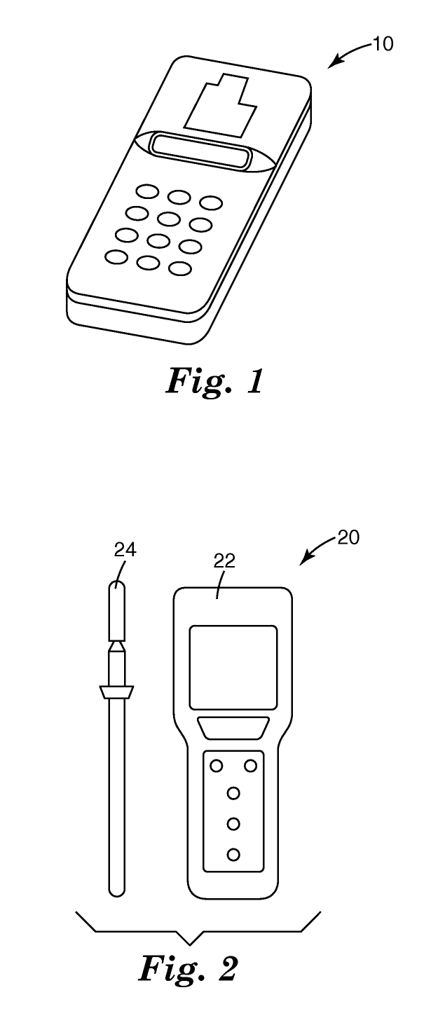

Two suitable devices of the present disclosure are illustrated in FIGS. 1 and 2. FIG. 1 shows a device 10, which is suitable as a hand-held device or a countertop device. Device 10 includes well known features, such as buttons for inputting information (e.g., the composition of the oil), appropriate means to provide radiation and appropriate means to measure the fluorescence, electronics that compare the measured level to a threshold, and a display for the user to read the results. A database of threshold levels may be stored within a memory or microprocessor in device 10. Device 10 may be battery powered or have an electric cord.

In this embodiment, device 10 is a non-contact, optical sensor, configured for irradiating the oil sample and measuring the fluorescence without contacting the oil. If a countertop unit, and oil sample could be brought to device 10, such as in a beaker or vial. If a hand-held unit, device 10 could be brought to the oil (e.g., the vat of hot oil) in close enough proximity to irradiate and measure the results.

A second device 20 is illustrated in FIG. 2. Device 20 can be a hand-held device or a countertop device, having a configuration to physically contact the oil sample. This device 20 includes a meter 22 and a sample receiver 24, which is operably engageable with meter 22. To use device 20, a sample of oil would be placed in sample receiver 24, for example by a swab, tube or pipette at least partially receivable within receiver 24. An additive, such as a fluorescent marker, may be present within receiver 24 or may be added after the oil sample. Receiver 24 may be inserted into or against meter 22, which would irradiate and measure the sample.

Meter 22 includes well known features, such as buttons for inputting information (e.g., the composition of the oil), appropriate means to provide radiation and appropriate means to measure the fluorescence, electronics that compare the measured level to a threshold, and a display for the user to read the results.

Device 20 is configured to contact a sample of oil removed from a larger batch. Other embodiments of devices for measuring oil quality according to this disclosure may contact the oil sample, without having to remove the sample from a larger batch. For example, a probe operable connected to a meter may be used.

### EXAMPLES

The invention is further illustrated in the following illustrative examples, in which all parts and percentages are by weight unless otherwise indicated.

Examples 1-14, below, show that when a fluorescent marker is used, fresh oil had a higher intensity than the used oil, demonstrating more non-polar constituents present in the oil. Examples 15-17, below, show that there is an increase in the autofluorescence of oil, without fluorescent marker dye, as the use of the oil increases.

Several fluorescent marker dyes (identified below) from Molecular Probes Inc. of Eugene, Oreg. were obtained. A solution of 1 mg/ml of each fluorescent dye was made win dimethyl sulfoxide (DMSO). 300 μl of each dye solution was further diluted in 3 ml fresh canola oil and mixed thoroughly.

Four samples of canola oil (fresh, used 1 week, used 10 days, used 2 weeks) were obtained. 30 μl of the dye solution was added to 3 ml of the oil being tested in the example and mixed thoroughly.

| Example | oil | dye |
|---|---|---|
| 1 | fresh | A |
| 2 | 10-day | A |
| 3 | fresh | B |
| 4 | 10-day | B |
| 5 | 2-week | B |
| 6 | fresh | C |
| 7 | 2-week | C |
| 8 | fresh | D |
| 9 | 2-week | D |
| 10 | fresh | E |
| 11 | 2-week | E |
| 12 | fresh | F |
| 13 | 2-week | F |
| 14 | fresh | none |
| 15 | 1 week | none |
| 16 | 10 days | none |

Fluorescent Marker Dye

A: 4,4-difluoro-1,3,5,7-tetramethyl-4-bora-3a,4a-diaza-s-indacene (commercially available as BODIPY® 505/515)

B: 6-acryloyl-2-dimethylaminonaphthalene (acrylodan)

C: 1-anilinonaphthalene-8-sulfonic acid (1,8-ANS)

D: 1,3-bis-(1-pyrenyl) propane

E: 6-dodecanoyl-2-dimethylaminormaphthalene (laurdan)

F: 9,10-bis-(N,N-dimethylaminomethyl)anthracene

Fluorescence spectra were measured, with the excitation wavelength corresponding to each dye, using a Fluorlog fluorimeter (from Horiba Jobin Yvon, Edison N.Y.).

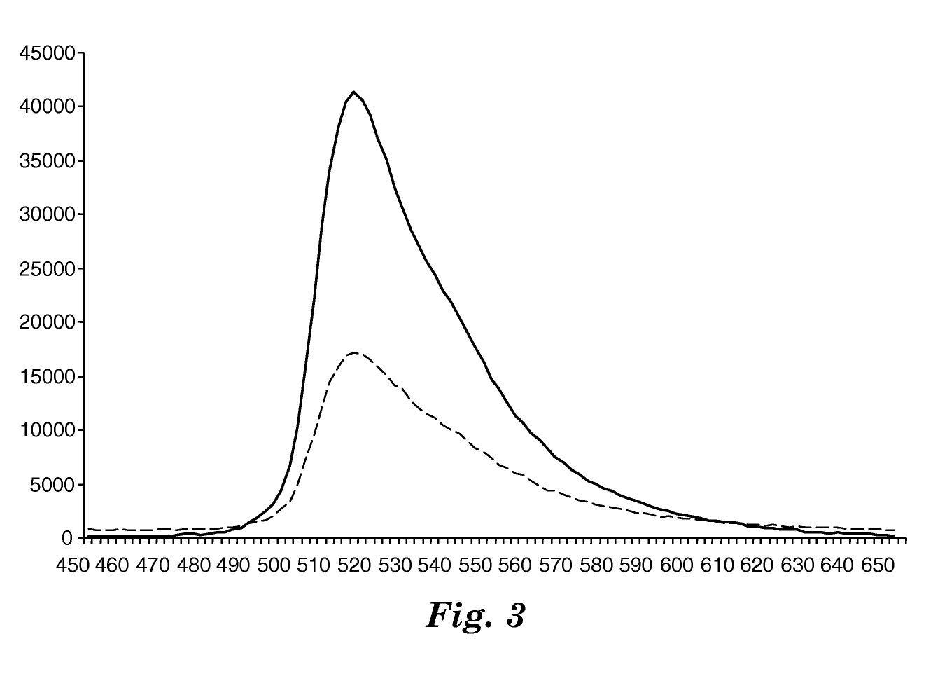

The fluorescence spectra of Examples 1 and 2 are shown in FIG. 3. This shows that the fresh canola oil, with a fluorescent marker, had a higher intensity than the used oil, demonstrating more non-polar constituents present in the oil.

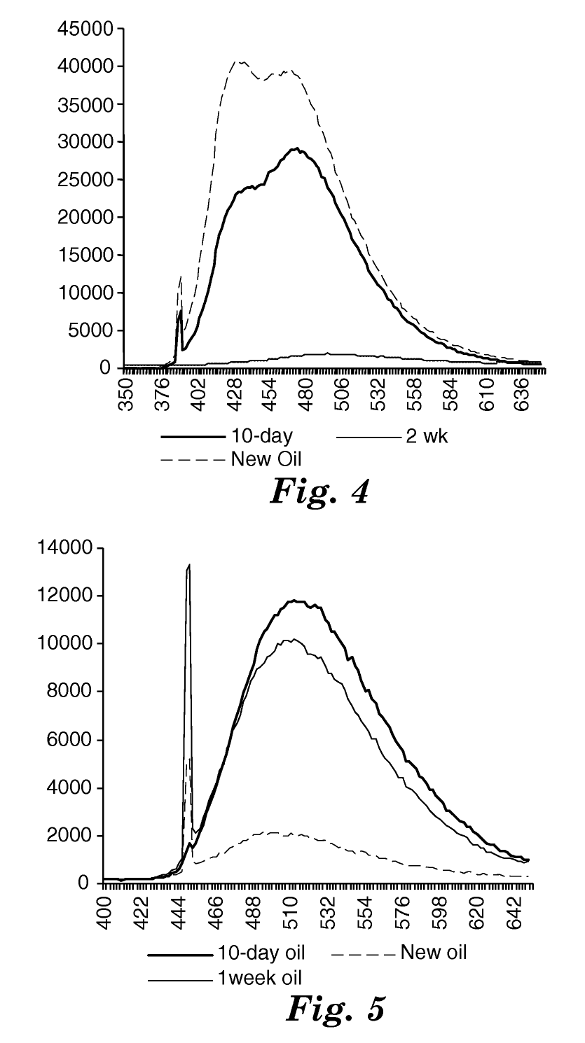

The fluorescence spectra of Examples 3, 4 and 5 are shown in FIG. 4. This shows that the fresh canola oil, with a fluorescent marker, had a higher intensity than the 10-day used oil, which had a higher intensity than the 2 week used oil, demonstrating that the fresher oil had more non-polar constituents present in the oil.

For each of the other sets of samples (i.e., Examples 6 and 7, Examples 8 and 9, Examples 10 and 11, and Examples 12 and 13) the fresher oil had a higher intensity than the older oils, demonstrating that the fresher oil had more non-polar constituents.

Examples 14, 15 and 16 included no fluorescent marker dye. The fluorescence spectra of Examples 14, 15 and 16 are shown in FIG. 5.

Examples 1-13, using a fluorescent marker dye, demonstrate that polarity sensitive florescent dye show drastic decrease in fluorescence intensity when used in old frying oil as compared to when used in fresh oil.

Examples 14-16 show that there is an increase in the autofluorescence of canola oil, even without fluorescent marker dye, as use (e.g., frying) of the oil continues.

Examples 17-97 show a correlation between the fluorescence of oil as measured by ISO 8420 and an optical device at 520 nm.

For Examples 17-97, samples of cooking oil were obtained from various sources, described below. All oil samples were heated in a microwave oven to melt any solidified oil. 200 μl of each oil sample were transferred into a well of a 96-well plate while still warm. If any sample solidified, the entire 96-well plate was again heated before conducting the measurement.

Fluorescence of each oil sample was measured with a Tecan microplate fluorimeter operating at an excitation wavelength of 470 nm and an emission wavelength of 520 nm. Polar content of each oil sample was measured according to ISO Standard 8420.

For Examples 17-20, the oil was 30% hydrogenated rapeseed/26.5% sunflower/43.5% palm oil. For Examples 21-34, the oil was 40% palm oil/29% sunflower oil/20% high oleic sunflower oil/11% rapeseed. For Examples 35-55, the oil was 40% high oleic sunflower (having at least 70% oleic fatty acid)/30% palm oil/30% hydrogenated rapeseed. For Examples 56-59, the oil was low TFA (Trans Fatty Acids) oil. For Example 60, the oil was 100% palm oil. For Examples 61-82, the oil was 100% high oleic sunflower oil. For Examples 83-89, the oil was 100% hydrogenated rapeseed oil. For Examples 90-97, the oil was a mix of high oleic sunflower oil, rapeseed oil, and grapeseed oil.

| | | | Fluorescence at |
|---|---|---|---|
| Example | Food Type | ISO 8420 (% PC) | 520 nm |
| 17 | Chicken | 18.8 | 16922.0 |
| 18 | Nuggets | 19.1 | 14774.0 |
| 19 | Fish | 20.3 | 16820.0 |
| 20 | Apple Pie | 19.3 | 11360.0 |
| 21 | French Fries | 19.6 | 16540.0 |
| 22 | French Fries | 14.1 | 13373.0 |
| 23 | French Fries | 14.5 | 13692.0 |
| 24 | French Fries | 18.6 | 70.0 |
| 25 | F. Fries/Chicken | 16.0 | 16007.0 |
| 26 | F. Fries/Chicken | 15.8 | 19539.0 |
| 27 | F. Fries/Chicken | 14.0 | 12210.0 |
| 28 | F. Fries/Chicken | 18.1 | 20379.0 |
| 29 | French Fries | 5.2 | 3035.0 |
| 30 | French Fries | 25.8 | 20852.0 |
| 31 | French Fries | 33.6 | 24616.0 |
| 32 | French Fries | 35.9 | 28582.0 |
| 33 | French Fries | 25.5 | 22909.0 |
| 34 | F. Fries/Chicken | 25.2 | 24118.0 |
| 35 | French fries | 6.1 | 3170.0 |
| 36 | French fries | 41.1 | 42581.0 |
| 37 | French fries | 49.1 | 39648.0 |
| 38 | French fries | 44.0 | 41214.0 |
| 39 | French fries | 52.9 | 34478.0 |
| 40 | French fries | 31.1 | 38490.0 |
| 41 | French fries | 35.9 | 35871.0 |
| 42 | French fries | 7.2 | 4158.0 |
| 43 | French fries | 8.8 | 8108.0 |
| 44 | French fries | 17.5 | 23827.0 |
| 45 | French fries | 22.2 | 31172.0 |
| 46 | French fries | 27.3 | 37459.0 |
| 47 | French fries | 31.2 | 41322.0 |
| 48 | French fries | 39.2 | 42731.0 |
| 49 | Nuggets | 4.6 | 2656.0 |
| 50 | Nuggets | 10.3 | 11698.0 |
| 51 | Nuggets | 12.0 | 16092.0 |
| 52 | Nuggets | 14.4 | 18626.0 |
| 53 | Nuggets | 16.0 | 22849.0 |
| 54 | Nuggets | 23.3 | 27012.0 |
| 55 | Nuggets | 22.4 | 30190.0 |
| 56 | French Fries | 27.0 | 27743.0 |
| 57 | French Fries | 8.3 | 3216.0 |
| 58 | Apple Pies | 8.8 | 1875.0 |
| 59 | Apple Pies | 30.5 | 24118.0 |
| 60 | Chicken Wings | 13.4 | 5382.0 |
| 61 | Nuggets | 14.5 | 27233.0 |
| 62 | Nuggets | 19.3 | 31238.0 |
| 63 | French Fries | 11.5 | 11934.0 |
| 64 | French Fries | 11.6 | 11581.0 |
| 65 | Potatoes | 11.8 | 14023.0 |
| 66 | Potatoes | 11.7 | 11065.0 |
| 67 | Fish | 13.7 | 30875.0 |
| 68 | Fish | 13.6 | 29040.0 |
| 69 | Chicken | 20.5 | 31431.0 |
| 70 | Chicken | 16.8 | 26277.0 |
| 71 | Nuggets | 6.3 | 10554.0 |
| 72 | French Fries | 5.6 | 6108.0 |
| 73 | Potatoes | 9.2 | 7117.0 |
| 74 | Fish | 7.1 | 12485.0 |
| 75 | Chicken | 8.4 | 5886.0 |
| 76 | French Fries | 3.6 | 2747.0 |
| 77 | French Fries | 4.6 | 4940.0 |
| 78 | French Fries | 9.3 | 11023.0 |
| 79 | French Fries | 13.1 | 18046.0 |
| 80 | French Fries | 18.6 | 27627.0 |
| 81 | French Fries | 18.2 | 28392.0 |
| 82 | French Fries | 18.5 | 29058.0 |
| 83 | Chicken | 9.6 | 25336.0 |
| 84 | Spiced Chicken | 16.6 | 27124.0 |
| 85 | French Fries | 16.9 | 20995.0 |
| 86 | Chicken | 12.7 | 27659.0 |
| 87 | Spiced Chicken | 9.1 | 19930.0 |
| 88 | French Fries | 21.3 | 28815.0 |
| 89 | French Fries | 19.1 | 28136.0 |
| 90 | French Fries | 13.9 | 22401.0 |
| 91 | French Fries | 15.7 | 24801.0 |
| 92 | French Fries | 14.6 | 19836.0 |
| 93 | French Fries | 11.5 | 16843.0 |
| 94 | French Fries | 8.1 | 7335.0 |
| 95 | French Fries | 10.4 | 11314.0 |
| 96 | French Fries | 9.1 | 17432.0 |
| 97 | French Fries | 10.3 | 18111.0 |

When graphed, a correlation of fluorescent signal to the total polar content (as determined by ISO 8420) is seen.

The above specification and examples are believed to provide a complete description of the manufacture and use of particular embodiments of the invention. Because many embodiments of the invention can be made without departing from the spirit and scope of the invention, the true scope and spirit of the invention reside in the broad meaning of the claims hereinafter appended.

What is claimed is:

1. A method of determining the quality of oil, the method comprising:
irradiating the oil at a wavelength of about 470 nm;
measuring a level of fluorescence of the oil at a wavelength of about 520 nm; and
comparing the measured fluorescence level to a predetermined threshold level.

2. The method of claim 1 wherein the oil has a composition and the predetermined threshold level is dependent on the oil composition.

3. The method of claim 2 further comprising discarding the oil if the measured fluorescence level exceeds the predetermined threshold level.

4. The method of claim 2 further comprising treating the oil for reuse using mechanical, physical or chemical means.

5. The method of claim 1 further comprising removing a sample of the oil from a larger volume of the oil prior to irradiating the oil.

6. The method of claim 5 further comprising adding a marker to the oil after removing a sample and prior to irradiating the oil.

7. The method of claim 5 further comprising adding the oil sample to a marker after removing a sample and prior to irradiating the oil.

8. The method of claim 1, wherein the step of measuring the level of fluorescence comprises correlating to a level of polar components in the oil.

9. A method of determining the quality of cooking oil, the method comprising:
irradiating the oil at a first wavelength;
measuring a level of fluorescence of the oil at a second wavelength different than the first wavelength; and
comparing the measured fluorescence level to a predetermined threshold level.

10. The method of claim 9, wherein the first wavelength is about 470 nm and the second wavelength is about 520 nm.

11. The method of claim 9, wherein the method is performed by an optical device.

12. The method of claim 9, wherein the method is performed by a device that contacts the oil.

13. The method of claim 9, wherein the step of measuring the level of fluorescence comprises correlating to a level of polar components in the oil.

14. A hand-held device for real-time measurement of the quality of oil, the device comprising:
means for irradiating the oil at a first wavelength;
means for measuring a level of fluorescence of the oil at a second wavelength; and
a display.

15. The device of claim 14, wherein
the means for irradiating the oil irradiates the oil at about 470 nm; and
the means for measuring the level of fluorescence measures at about 520 nm.

16. The device of claim 14, wherein
the means for irradiating the oil irradiates the oil with blue light.
the means for measuring the level of fluorescence measures with green light.

17. The device of claim 14 wherein the means for measuring the level of fluorescence is an optical detector.

18. The device of claim 14 further comprising a database comprising a plurality of threshold levels.

19. The device of claim 18, wherein the threshold levels are dependent on a composition of the oil.

20. The device of claim 14 further comprising a data communication connection.

21. The device of claim 20, wherein the device is connected to a data network for storing, retrieving and updating data corresponding to the quality of the oil.

22. The device of claim 14 further comprising a collection device and a receptacle for the collection device, the receptacle in optical contact with the means for irradiating the oil and the means for measuring the level of fluorescence.

23. The device of claim 22 wherein the collection device comprises at least one of a tube, a swab, or a pipette tip.

24. The device of claim 22 wherein the receptacle includes a marker for fluorescence therein.

25. The device of claim 14 further comprising a probe operably connected to the means for irradiating the oil and the means for measuring the level of fluorescence.

26. The device of claim 14 further comprising an integrated system for sample collection, data measurement and data management.
